(19) United States
(12) Patent Application Publication
(10) Pub. No.: US 2009/0208572 A1
(43) Pub. Date: Aug. 20, 2009

(54) ORAL CONTROLLED RELEASE TABLET

(75) Inventors: Nitin Bhalachandra Dharmadhikari, Mumbai (IN); Yashoraj Rupsinh Zala, Mumbai (IN)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SUN PHARMA ADVANCED RESEARCH COMPANY LIMITED, Mumbai (IN)

(21) Appl. No.: 12/372,161

(22) Filed: Feb. 17, 2009

(30) Foreign Application Priority Data
Feb. 15, 2008 (IN) 339/MUM/2008

(51) Int. Cl. A61K 9/24

(52) U.S. Cl. 424/472

(57) ABSTRACT

A method of reducing the risk of alcohol-induced dose-dumping of a therapeutically active ingredient comprising administering to human subjects who have ingested alcohol an oral controlled release tablet; said tablet comprising:
a core comprising
   an upper compressed layer comprising a swelling agent, and
   a lower compressed layer comprising at least one therapeutically active ingredient, and pharmaceutically acceptable excipient wherein at least one excipient is a release rate controlling excipient and wherein the percent by weight of excipients that are soluble in alcohol does not exceed 35% by weight of the layer and;
a coating surrounding the said core, the coating comprising a polymer insoluble in an aqueous medium comprising from 0% v/v to 40% v/v of alcohol, whereby upon contact with aqueous gastrointestinal fluids, the upper compressed layer swells to cause removal of the coating from the upper surface of the upper compressed layer and then said upper layer disintegrates allowing the release of the active ingredient from the defined surface area of the upper surface of said lower compressed layer with the coating covering its bottom and side surfaces.

### FIELD OF THE INVENTION

The present invention relates to a method of reducing the risk of alcohol-induced dose-dumping of a therapeutically active ingredient.

### BACKGROUND OF THE INVENTION

Oral controlled release drug delivery systems contain at least twice the amount of drug compared to the conventional dosage forms and therefore require careful design to prevent rapid release of the dosage amount of the drug. This unintended, rapid drug release of a significant fraction of the drug contained in the controlled release drug delivery systems in a shorter period of time may be referred to as ‘dose dumping’. Although the dose dumping caused by the presence of food has been addressed for about twenty years by regulatory bodies, the dose dumping caused by alcohol consumption has only recently received attention. In 2005, several drugs were withdrawn from the market because of the effects of ethanol on the controlled release formulations. For instance, the United States Food and Drug Administration (FDA) asked Purdue Pharma to withdraw Palladone® (hydromorphone hydrochloride) extended release capsules from the market (FDA press release of Jul. 13, 2005.) Food and Drug Administration, USA is now assessing criteria for defining the regulatory procedure for distinguishing between the vulnerable (prone to dose-dumping) and rugged (not prone to dose-dumping) controlled release products. In vitro dissolution test in 40% v/v ethanol in either water or 0.1 N HCL was used to investigate “dose dumping.” (FDA's ACPS Meeting, October 2005, Awareness Topic: Mitigating the Risks of Ethanol induced dose dumping for oral sustained/controlled release dosage forms, by Robert Meyer, Ajaz Hussain, Office of New Drugs and Office of Pharmaceutical Science Centre for Drug Evaluation and Research, FDA).

Several attempts are being made after the awareness of the alcohol-induced dose dumping in sustained release formulations. For example, PCT publication, WO2007016563 disclosed a modified release oral dosage form, comprising (a) a therapeutic agent and; (b) an alcohol insoluble coating wherein in between 0% and 35% of the therapeutic agent is released from the dosage form in vitro after 60 minutes in the presence of 40% alcohol at pH 1.2.

PCT publication, WO2007053698 A2 (Alza Corp.) discloses a method of reducing adverse effects associated with alcohol-induced dose dumping in patients who are orally receiving sustained release hydromorphone comprising; providing a sustained dosage form which comprises a dose of hydromorphone; and administering the dosage form to a patient wherein when tested using an in vitro test method that employs a test medium that comprises aqueous alcohol at a concentration of about 20% volume/volume, the dosage form releases less than or equal to about 50 weight percent of the dose of hydromorphone in a period of about 2 hours following initiation of the in vitro test method.

PCT publication WO2007078895 (Biovail Laboratories) relates to a specific type of controlled and modified release dosage form containing tramadol or at least one pharmaceutically acceptable salt, enantiomer, or metabolite-thereof, that passes the specific pharmacokinetic properties and which desirably are not subject to the dose dumping, e.g, induced by food or alcohol.

Another prior art United States Patent application number US20060193911 (hereinafter referred to as application number '3991) discloses a controlled release oral solid dosage form comprising: a matrix comprising a therapeutically effective amount of Venlafaxine, an active metabolite of Venlafaxine or a pharmaceutically acceptable salt thereof, dispersed in a cross linked agent, said matrix providing a controlled release of Venlafaxine, active metabolite of Venlafaxine, or salt thereof to provide 24 hour therapeutic plasma levels after oral administration to human patients. The '3991 application relates to controlled release dosage forms containing therapeutically effective amount of Venlafaxine, an active metabolite of venlafaxine which are resistant to alcohol induced dose dumping.

United States Patent application US20070264346 discloses an oral pharmaceutical or dietetic form comprising microparticles of the reservoir type for the modified release of at least one active principle (AP), characterized in that it is resistant to immediate dumping of the dose of the AP in the presence of alcohol.

PCT Publication namely, WO 2007103293 A2 discloses a method of preventing dose-dumping of a drug in the presence of ethanol comprising; providing a patient likely to consume ethanol while being treated with the drug an effective amount of the drug in the form of an ethanol-resistant sustained release formulation comprising: the drug; and a sustained release delivery system, the delivery system comprising at least one heteropolysaccharide gum, at least one homopolysaccharide gum and at least one pharmaceutical diluent, wherein the ethanol-resistant sustained release formulation essentially retains its sustained release dissolution profile in the presence of ethanol.

Controlled release or sustained release drug delivery systems may be designed so as to be administered twice a day or systems may be designed so as to be administered once a day. The higher the dose in a single unit dosage form, the greater is the harm if the dose is immediately released i.e dumped into the gastrointestinal fluid. Generally, oral controlled release drug delivery systems may release the drug over a period of about 8 to about 20 hours depending on the pharmacokinetics of the drug. Therefore, when tested in vitro, if a significant fraction, for example, more than 80% of the dose is released within half of this duration i.e in about 4 to about 10 hours, it may be a cause of concern for a patient. Dose dumping generally includes such rapid release of a large fraction of the dose. There is no accepted definition of dose-dumping that could be applied for various therapeutically active ingredients. Instead, any release that occurs at a significantly enhanced rate so as to enhance the incidence of undesirable side effects or adverse effects can be considered as “dose-dumbing”. As referred to herein, the term “dose-dumping” will mean release of more than 80% in four hours or more than 40% in two hours when tested by in vitro dissolution.

There is a need to provide a method of reducing the risk of dose-dumping by administering an oral controlled drug delivery system that resist dose-dumping under various conditions for example, fasted or fed state of the human subjects. Particularly, there is a need to provide a method of reducing the risk of alcohol induced dose-dumping by administering an oral controlled drug delivery systems that resist alcohol-induced dose-dumping.

We have now found a method of reducing the risk of alcohol-induced dose-dumping by administering selective embodiments of the oral controlled drug delivery systems prepared according to our pending PCT publications, namely WO2005039481, WO2006123364 and Indian Patent application number 2374/MUM/2007.

### OBJECTS OF THE INVENTION

The object of the present invention is to provide a method of reducing the risk of alcohol induced dose dumping.

Another object of the present invention is to provide a method of reducing the risk to patients associated with ethanol induced dose dumping, either due to safety issues or diminished efficacy or both.

It is yet another object of the present invention to provide a controlled release tablet comprising active ingredient that releases the active ingredient in a controlled manner when the tablet is administered with alcohol.

### SUMMARY OF THE INVENTION

The present invention provides a method of reducing the risk of alcohol-induced dose-dumping of a therapeutically active ingredient comprising administering to human subjects who have ingested alcohol; an oral controlled release tablet said tablet comprising:
a core comprising
   an upper compressed layer comprising a swelling agent, and
   a lower compressed layer comprising at least one therapeutically active ingredient, and pharmaceutically acceptable excipient wherein at least one excipient is a release rate controlling excipient and wherein the percent by weight of excipients that are soluble in alcohol does not exceed 35% by weight of the layer and;
a coating surrounding the said core, the coating comprising a polymer insoluble in an aqueous medium comprising from 0% v/v to 40% v/v of alcohol,
   whereby upon contact with aqueous gastrointestinal fluids, the upper compressed layer swells to cause removal of the coating from the upper surface of the upper compressed layer and then said upper layer disintegrates allowing the release of the active ingredient from the defined surface area of the upper surface of said lower compressed layer with the coating covering its bottom and side surfaces.

The present invention also relates to a method of reducing the risk of alcohol-induced dose-dumping of a therapeutically active ingredient comprising administering to human subjects who have ingested alcohol an oral controlled release tablet; said tablet comprising: a core comprising
an upper compressed layer comprising a swelling agent, and
a middle compressed layer comprising at least one therapeutically active ingredient, and pharmaceutically acceptable excipient wherein at least one excipient is a release rate controlling excipient and wherein the total amount of excipients that are soluble in alcohol does not exceed 35% by weight of the layer and;
a bottom compressed layer comprising a swelling agent,
coating surrounding the said core, the coating comprising a polymer insoluble in an aqueous medium comprising from 0% v/v to 40% v/v of alcohol content,
   whereby upon contact with aqueous gastrointestinal fluids, the upper compressed layer and the bottom compressed layer swell to cause removal of the coating from the upper surface of the upper compressed layer and the lower surface of the bottom compressed layer and then said upper layer and the said bottom layer disintegrate, allowing the release of the active ingredient from the defined surface area of the upper and lower surface of said middle compressed layer with the coating covering its side surfaces.

### DESCRIPTION OF THE FIGURES AND DRAWINGS

Many aspects of the invention can be better understood with reference to the following figures. The figures only represent one of the embodiments of the present invention. The embodiments are meant only for the purpose of illustration of the present invention. The components in the drawings are not necessarily to scale, emphasis instead being placed upon clearly illustrating the principles of the present invention.

Different embodiments of the present invention are diagrammatically represented in FIG. 1 to FIG. 5.

FIG. 1: Different parts of the coated tablet are labeled as below
1. upper compressed layer comprising a swelling agent
2. lower compressed layer comprising therapeutically active ingredient
3. coating comprising a polymer insoluble in an aqueous medium of 0% to 40% v/v of alcohol content
4. laser drilled passageway
5. aqueous environment, arrow marks depicting entry of water through the passageway
(a) upper surface of the lower compressed layer
(b) bottom surface of the lower compressed layer
(c) side surface of the lower compressed layer

FIG. 1(A) represents a bilayer tablet surrounding a coated with a pre formed passageway on its upper surface.

FIG. 1(B) represents the stage where the tablet comes in contact with the aqueous environment. Upon contact with the aqueous environment (5), there is a rapid ingress of water through the laser drilled passageway on the upper surface of the tablet.

FIG. 1(C) represents next stage where upon ingress of aqueous fluids, the upper compressed layer swells rapidly. The swelling of the excipients exerts a pressure from inside on surface of coating having the laser drilled passageway. This causes removal of the coating from the upper surface of the tablet. The upper compressed layer then starts disintegrating.

FIG. 1(D) represents the stage where the upper compressed layer is completely disintegrated leaving the lower compressed layer exposed to the aqueous environment allowing the release of the therapeutic active ingredient from the defined surface area of the upper surface of said lower compressed layer with the coating covering its bottom and side surfaces.

FIG. 2 represents one embodiment where core is bilayer and the coating comprises an enteric polymer and a polymer insoluble in an aqueous medium of 0% v/v to 40% v/v of alcohol content.

Different parts of the coated tablet are labeled as below
1. upper compressed layer comprising a swelling agent
2. lower compressed layer comprising therapeutically active ingredient
3. coating comprising an enteric polymer and a polymer insoluble in an aqueous medium of 0% v/v to 40% v/v of alcohol content
4. in situ formed passageways on the upper surface of the upper compressed layer
5. aqueous environment, arrow marks depicting entry of water through the passageway
6. microporous channels
7. upper surface of the upper compressed layer
(a) upper surface of the lower compressed layer
(b) bottom surface of the lower compressed layer
(c) side surface of the lower compressed layer

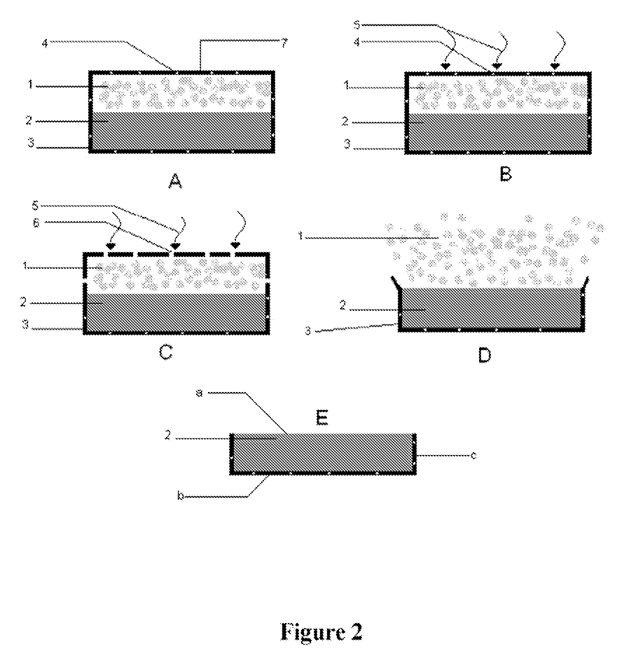

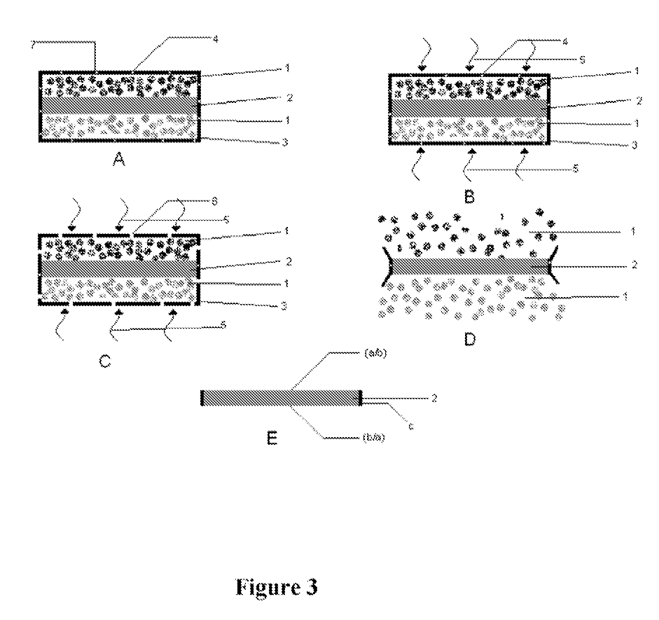

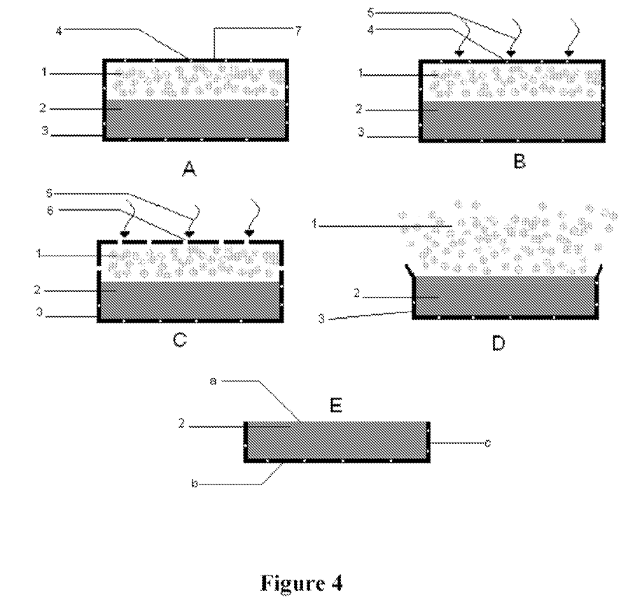

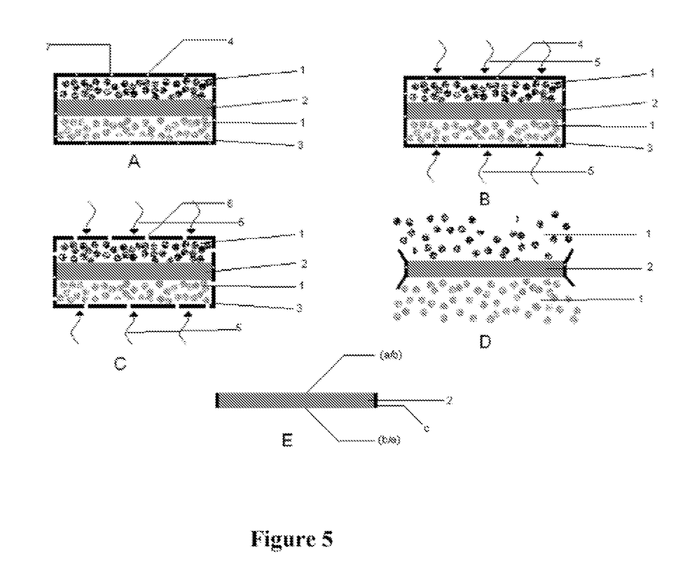

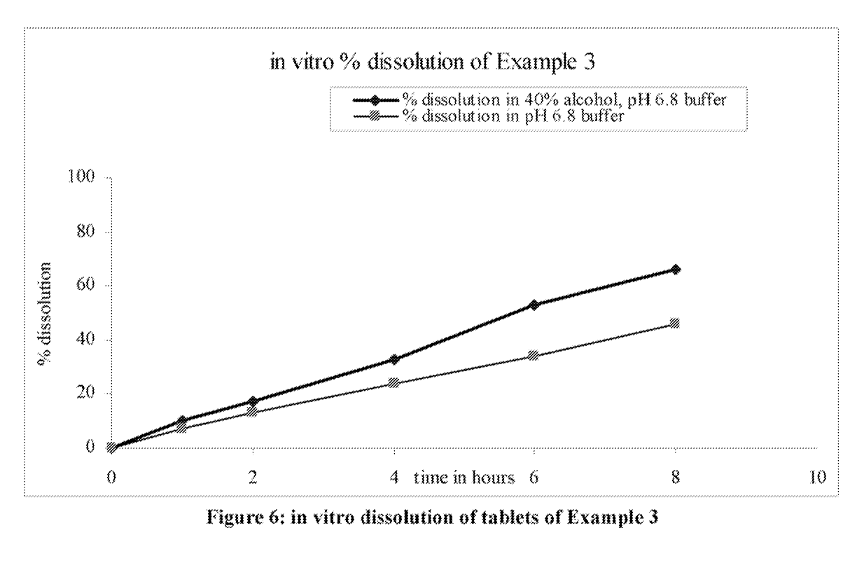

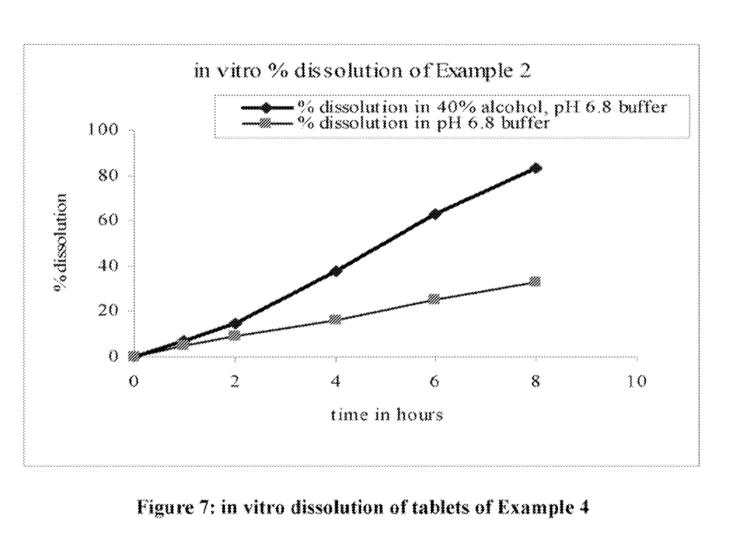

FIG. 2(A) represents the bilayer tablet coated with a mixture of polymer insoluble in aqueous medium of 0% v/v to about 40% v/v alcohol and enteric polymer.

FIG. 2(B) represents the stage where the coated tablet comes in contact with the aqueous environment. FIG. 2(C) represents a stage where, upon contact with the aqueous environment (5), microporous channels are formed as the enteric polymer dissolves allowing ingress of aqueous fluids into the tablet.

FIG. 2(D) represents next stage where upon ingress of aqueous environment, the upper compressed layer swells rapidly. The swelling of the excipients exerts a pressure on upper surface of the coating. This causes removal of the coating from the upper surface of the tablet. The upper compressed layer then starts disintegrating.

FIG. 2(E) represents the stage where the upper compressed layer is completely disintegrated leaving the lower compressed layer exposed to the aqueous environment allowing the release of the therapeutically active ingredient to occur from the defined surface area of the upper surface of said lower compressed layer with the coating covering its bottom and side surfaces.

FIG. 3 represents one embodiment where the core is trilayered and the coating comprises an enteric polymer and a polymer insoluble in an aqueous medium of 0% v/v to 40% v/v of alcohol content.

Different parts of the coated tablet are labeled as below
1. upper/lower compressed layer comprising a swelling agent
2. middle compressed layer comprising therapeutically active ingredient
3. coating comprising an enteric polymer and a polymer insoluble in an aqueous medium of 0% v/v to 40% v/v of alcohol
4. in situ formed passageway
5. aqueous environment, arrow marks depicting entry of water through the passageway
6. microporous channels
7. upper surface of the upper compressed layer
(a) upper surface of the lower compressed layer
(b) bottom surface of the lower compressed layer
(c) side surface of the lower compressed layer

FIG. 3(A) is a trilayered tablet coated with a enteric polymer and a polymer insoluble in aqueous medium of 0% to about 40% v/v alcohol content.

FIG. 3(B) represents the stage where the coated tablet comes in contact with the aqueous environment. FIG. 3(C) represents the stage where upon contact with the aqueous environment (5), microporous channels are formed as the enteric polymer dissolves allowing ingress of fluids into the tablet.

FIG. 3(D) represents next stage where upon ingress of aqueous fluids, the upper and the lower compressed layers swell rapidly. The swelling of the excipients exerts a pressure on surface of coating which are in immediate vicinity of the compressed layers having swelling agents. This causes removal of the coating from the upper and lower surfaces of the tablet. The upper compressed layer and lower compressed layer starts disintegrating.

FIG. 3(E) represents the stage where the upper and the lower compressed layers are completely disintegrated leaving the middle compressed layer exposed to the aqueous environment allowing the release of the therapeutically active ingredient from the defined surface area of the upper and the lower surface of said lower compressed layer with the coating covering its side surfaces.

FIG. 4 represents one embodiment where the tablet is coated with a coating comprising leachable component and a polymer insoluble in an aqueous medium of 0% to about 40% v/v of alcohol content.

Different parts of the coated tablet are labeled as below
1. upper compressed layer comprising a swelling agent
2. lower compressed layer comprising therapeutically active ingredient
3. coating comprising a water soluble leachable component and a polymer insoluble in an aqueous medium of 0% v/v to 40% v/v of alcohol content
4. in situ formed passageway
5. aqueous environment, arrow marks depicting entry of water through the passageway
6. microporous channels
7. upper surface of the upper compressed layer
(a) upper surface of the lower compressed layer
(b) bottom surface of the lower compressed layer
(c) side surface of the lower compressed layer
FIG. 4(A) represents the tablet according to one embodiment of the present invention wherein the core is bilayer and the coating comprises leachable components and polymer insoluble in an aqueous medium of 0% v/v to 40% v/v of alcohol content.

FIG. 4(B) represents the stage where the coated tablet comes in contact with the aqueous environment. FIG. 4(C) represents the stage where upon contact with the aqueous environment (5), microporous channels are formed as leachable components dissolve allowing ingress of aqueous fluids into the tablet.

FIG. 4(D) represents next stage where upon ingress of aqueous environment, the upper compressed layer swells rapidly. The swelling of the excipients exerts a pressure on upper surface of coating. This causes removal of the coating from the surface of the tablet which is having the compressed layer in its immediate vicinity. The upper compressed layer then starts disintegrating.

FIG. 4(E) represents the stage where the upper compressed layers are completely disintegrated leaving the lower compressed layer exposed to the aqueous environment allowing the release of the therapeutically active ingredient from the defined surface area of the upper surface of said lower compressed layer with the coating covering its bottom and side surfaces.

FIG. 5 represents one embodiment where the tablet is coated with a coating comprising leachable component and a polymer insoluble in an aqueous medium of 0% v/v to 40% v/v of alcohol content.

Different parts of the coated tablet are labeled as below
1. upper/lower compressed layer comprising a swelling agent
2. middle compressed layer comprising therapeutically active ingredient
3. coating comprising leachable component and a polymer insoluble in an aqueous medium of O % to about 40% v/v of alcohol content
4. in situ formed passageway
5. aqueous environment, arrow marks depicting entry of water through the passageway
6. microporous channels
7. upper surface of the upper compressed layer
(a) upper surface of the lower compressed layer
(b) bottom surface of the lower compressed layer
(c) side surface of the lower compressed layer

FIG. 5(A) represents the tablet according to one embodiment of the present invention wherein the core is trilayered and the coating comprises leachable components and polymer insoluble in an aqueous medium of 0% v/v to 40% v/v of alcohol content.

FIG. 5(B) represents the stage where the coated tablet comes in contact with the aqueous environment. FIG. 5(C) represents the stage where upon contact with the aqueous environment (5), microporous channels are formed as the leachable component dissolve in water causing ingress of aqueous fluids into the tablet.

FIG. 5(D) represents next stage where upon ingress of aqueous fluids, the upper and the lower compressed layer swells rapidly. The swelling of the excipients exerts a pressure on surface of coating which are in immediate vicinity of the compressed layers having swelling agents. This causes removal of the coating from the upper and lower surfaces of the tablet. The upper compressed layer and lower compressed layer then starts disintegrating.

FIG. 5(E) represents the stage where the upper and the lower compressed layers are completely disintegrated leaving the middle compressed layer exposed to the aqueous environment allowing the release of the therapeutically active ingredient from the defined surface area of the upper and the lower surface of said middle compressed layer with the coating covering its side surfaces.

FIG. 6 depicts the % drug released in vitro dissolution when the tablets of example 3 were tested with or without alcohol in 6.8 phosphate buffer in type I apparatus rotating at a speed of 100 rpm

FIG. 7 depicts the % drug released in vitro dissolution when the tablets of example 4 were tested with or without alcohol in 6.8 phosphate buffer in type I apparatus rotating at a speed of 100 rpm.

### DETAILED DESCRIPTION OF THE INVENTION

Depending on the therapeutic indication and the therapeutic index of an active ingredient, dose dumping can pose a significant risk to patients, either due to safety issues or diminished efficacy or both. The present invention provides method of reducing such risks.

The present invention provides a method of reducing the risk of alcohol-induced dose-dumping of a therapeutically active ingredient comprising administering to human subjects who have ingested alcohol an oral controlled release tablet; said tablet comprising:
a core comprising
   an upper compressed layer comprising a swelling agent, and
   a lower compressed layer comprising at least one therapeutically active ingredient, and pharmaceutically acceptable excipient wherein at least one excipient is a release rate controlling excipient and wherein the percent weight of excipients that are soluble in alcohol does not exceed 35% by weight of the layer and;
a coating surrounding the said core, the coating comprising a polymer insoluble in an aqueous medium comprising from 0% v/v to 40% v/v of alcohol,
   whereby upon contact with aqueous gastrointestinal fluids, the upper compressed layer swells to cause removal of the coating from the upper surface of the upper compressed layer and then said upper layer disintegrates allowing the release of the active ingredient from the defined surface area of the upper surface of said lower compressed layer with the coating covering its bottom and side surfaces.

The present invention also provides a method of reducing the risk of alcohol-induced dose-dumping of a therapeutically active ingredient comprising administering to human subjects who have ingested alcohol an oral controlled release tablet; said tablet comprising:
a core comprising
   an upper compressed layer comprising a swelling agent, and
   a middle compressed layer comprising at least one therapeutically active ingredient, and pharmaceutically acceptable excipient wherein at least one excipient is a release rate controlling excipient and wherein the total amount of excipients that are soluble in alcohol does not exceed 35% by weight of the layer and;
   a bottom compressed layer comprising a swelling agent,
a coating surrounding the said core, the coating comprising a polymer insoluble in an aqueous medium comprising from 0% v/v to 40% v/v of alcohol content,
   whereby upon contact with aqueous gastrointestinal fluids, the upper compressed layer and the bottom compressed layer swell to cause removal of the coating from the upper surface of the upper compressed layer and the lower surface bottom compressed layer of the tablet and then said upper layer and the said bottom layer disintegrate, allowing the release of the active ingredient from the defined surface area of the upper and lower surface of said middle compressed layer with the coating covering its side surfaces.

As referred to herein, the term “dose-dumping” will mean release of more than 80% in four hours or more than 40% in two hours when tested by in vitro dissolution. The in vitro dissolution test is done in either aqueous medium or aqueous medium having pH 1.2, for example, 0. 1 N hydrochloric acid or pH 4.5 acetate buffer or pH 6.8 phosphate buffer, with 0% to about 40% alcohol using Type I or Type II USP dissolution apparatus at a suitable speed. Generally, the oral controlled release tablet is said to prevent or reduce the risk of alcohol induced dose dumping when the tablet does not release more than 80% of the therapeutically active ingredient in about four hours after initiation of in vitro dissolution test in about 10% to about 40% aqueous alcohol or when the tablet does not release more than 40% of the therapeutically active ingredient in about two hours after the initiation of in vitro dissolution in about 10% to about 40% aqueous alcohol.

The examples of the therapeutically active ingredient used in the oral controlled release tablet of the present invention include, but are not limited to, the following, viz. alcohol abuse preparations, drugs used for Alzheimer's disease, anesthetics, acromegaly agents, analgesics, antiasthmatics, anticancer agents, anticoagulants and antithrombotic agents, anticonvulsants, antidiabetics antiemetics, antiglaucoma, antihistamines, anti-infective agents, antiparkinsons, antiplatelet agents, antirheumatic agents, antispasmodics and anticholinergic agents, antitussives, carbonic anhydrase inhibitors, cardiovascular agents, cholinesterase inhibitors, treatment of CNS disorders, CNS stimulants, contraceptives, cystic fibrosis management, dopamine receptor agonists, endometriosis management, erectile dysfunction therapy, fertility agents, gastrointestinal agents, immunomodulators and immunosuppressives, memory enhancers, migraine preparations, muscle relaxants, nucleoside analogues, osteoporosis management, parasympathomimetics, prostaglandins, psychotherapeutic agents, sedatives, hypnotics and tranquilizers, drugs used for skin ailments, steroids and hormones.

We have found a reduced risk of alcohol induced dose dumping with the use of embodiments of tablets of our earlier inventions described in PCT publications, WO2005039481, WO2006123364 and Indian Patent application number 2374/MUM/2007, wherein such embodiments have less than 35% by weight of the alcohol soluble excipients in the compressed layer comprising at least one therapeutically active ingredient in the controlled release tablet. The term alcohol soluble as used herein means that at least one part by weight of the excipient dissolves in ten parts by weight of ethanol when stirred at room temperature for 8 hours. The amount of excipient dissolved in alcohol may be measured by any suitable analytical method.

Examples of alcohol soluble excipients that may be used in the compressed layer comprising therapeutically active ingredient and release rate controlling excipients, include, but are not limited to, citric acid, polyvinyl pyrrolidone, methacrylic acid copolymers and the like and mixtures thereof, acrylic acid polymer such as carboxyvinyl polymer (polyacrylic acid), cellulose acetate, for example, cellulose triacetate, cellulose diacetate, cetostearyl alcohol, dextrose, ethyl cellulose (ethoxy content more than 46.5%), fructose, certain viscosity grades of hydroxypropyl cellulose, malic acid, mannitol, polyethylene-propylene glycol copolymers for example poloxamer, polydextrose, polyoxyethylene alkyl ethers, polyoxyethylene sorbitan fatty acid esters, propylene glycol alginate of with certain degree of esterification, saccharin and its salts, stearic acid, tartaric acid and the like and mixtures thereof. The amount of excipients that are soluble in alcohol does not exceed 40% by weight of the compressed layer that comprising active ingredient and release rate controlling excipients. More particularly, the said amount of alcohol soluble excipients does not exceed 35% by weight of the said compressed layer.

The core of the tablets of the present invention comprises upper and/or compressed layer comprising swelling agent and the lower/middle compressed layer comprising the active ingredient and at least one excipient is a release rate controlling excipient. These layers occupy ‘separate regions’ in the core. By the term ‘separate regions’ as used herein means that the two layers occupy separate volumes, such that the two compositions are not substantially mixed together. A small amount of intermixing of the two layers may occur where the compositions come in contact with each other, for example, at the interface between the layers.

The compressed layer comprising therapeutically active ingredient comprises excipients that control the release of the active ingredient for prolonged period, for example for about more than about six hours, preferably eight hours. Such excipients are herein after referred to as “rate controlling excipients”. These rate controlling excipients used in the present invention may be selected from hydrophilic polymers such as methyl cellulose, hydroxypropyl methylcellulose, hydroxyethyl cellulose, hydroxypropyl cellulose, hydroxyethyl methylcellulose, carboxymethylcellulose and sodium carboxymethylcellulose; hydrophobic compounds such as ethyl cellulose, glycerol palmitostearate, beeswax, glycowax, castor wax, carnauba wax, glycerol monostearate, stearyl alcohol, glycerol behenic acid ester, cetyl alcohol, natural and synthetic glycerides, waxes, fatty acids, hydrophobic polyacrylamide derivatives, hydrophobic methacrylic acid derivatives; vinyl pyrrolidone polymers such as polyvinylpyrrolidone and copolymers of vinyl pyrrolidone and vinyl acetate; alkylene oxide homopolymers; gums of plant, animal, mineral or synthetic origin; and mixtures thereof. The rate controlling excipients may be used in an amount ranging from about 2% to about 99% by weight of compressed layer comprising the active ingredient.

One of the embodiments of the present invention uses hydroxypropylmethylcellulose (HPMC) having viscosity ranging from about 50 to about 25,000 mPa·sec as a release rate controlling excipient. Examples of the HPMC that may be used, include, but are not limited to, Methocel K4M, K15M and K100M and the like and mixture thereof. In this embodiment, preferably some or all of the HPMC polymers have a viscosity in the range of from 1000 to 25,000 mPa·sec. Preferably, HPMC having viscosity of 100,000 cps is used. The percentage of the hydroxypropyl methylcellulose may range from about 5% to 50% by weight of the compressed layer comprising a therapeutically active ingredient.

The swelling agent used in the upper or the bottom compressed layer according to the present invention is a material that swells but does not form a strong gel and thereby it favours disintegration of the said layer upon contact with the aqueous environment. Also it does not hinder in the disintegration of the layer. Any pharmaceutically acceptable material that meets such a functional requirement can be considered to be suitable as a swelling agent in the upper or bottom compressed layer. Any material that will swell but does not form a strong gel can be used in the present invention however preferably the swelling agent is selected from the group consisting of wicking agents, super disintegrants and mixtures thereof. Preferably the swelling agent is one that can swell upon imbibing water to at least twice its original volume or it may a material that enhances the swelling of other excipients that are capable of swelling. The swelling agents may be a mixture of at least one superdisintegrant or silicified microcrystalline cellulose with adjuvants that promote the swelling property. Such adjuvants include materials such as gas generating agents and osmogents. It is possible to use a wicking agent alone as the swelling agent. A wicking agent may be defined as any material with the ability to draw water into the matrix. A wicking agent can do this with or without swelling. Examples of wicking agents that may be used include, but are not limited to, colloidal silicon dioxide, kaolin, titanium dioxide, fumed silicon dioxide, alumina, low molecular weight polyvinylpyrrolidone, microcrystalline cellulose, bentonite, magnesium aluminum silicate (Veegum K) and the like and mixtures thereof. In one preferred embodiment, the wicking agent used in the oral controlled release tablet of the present invention includes, cellulose and cellulose derivatives, colloidal silicon dioxide, and mixtures thereof.

In one embodiment of the present invention, the upper or the bottom compressed layer comprises co-processed microcrystalline cellulose as a wicking agent. The microcrystalline cellulose is co-processed with silicon dioxide preferably colloidal silicon dioxide. Such a co-processed microcrystalline cellulose (silicified MCC) shows improved compressibility as compared to standard grades of microcrystalline cellulose. The silicified microcrystalline cellulose with varying amounts of silicon dioxide is commercially available under the brand name Prosolv®. Typically the colloidal silicon dioxide content is about 2% w/w. The most preferred embodiments of the present invention use silicified microcrystalline cellulose with 2% w/w of colloidal silicon dioxide. These are available commercially under the brand name Prosolv SMCC® 90 with a median particle size in the region of 90 μm and Prosolv SMCC® 50 with a median particle size in the region of 50 μm.

According to one embodiment of the present invention, the amount of silicified microcrystalline cellulose that may be used in the present invention may range from about 0.1% w/w to about 95% w/w, more preferably from about 1% to about 90% and most preferably from about 5% to about 80% by weight of the upper compressed layer.

The swelling agent of the present invention may be a super-disintegrant. Examples of super disintegrants that may be used are selected from the group comprising cross linked vinylpyrrolidone polymers such as crospovidone; cellulose and cellulose derivatives such as carboxyalkyl celluloses, low substituted hydroxypropyl cellulose, crosslinked carboxyalkylcellulose and their alkali salts; starch and starch derivatives such as pregelatinized starch, dried starch, sodium starch glycolate; resins such as polacrillin potassium (Amberlite IRP 88) and the like and mixtures thereof. The super disintegrants may be used in amount ranging from about 0% to about 80% by weight of the upper compressed layer and most preferably in an amount ranging from about 5% to about 30% by weight of the upper compressed layer.

Examples of gas generating agents that may be used as swelling agent include, but are not limited to, carbonates such as calcium carbonate, bicarbonates such as sodium or potassium bicarbonate, sulfites such as sodium sulfite, sodium bisulfite, or sodium metabisulfite, and the like. These salts may be used alone or in combination with an acid source as a gas generating couple. The acid source may be an edible organic acid, a salt of an edible organic acid, acidic components such as acrylate polymers, or mixtures thereof. Examples of organic acids that may be used include citric acid, malic acid, succinic acid, tartaric acid, fumaric acid, maleic acid, ascorbic acid, glutamic acid, and their salts, and mixtures thereof. The amount of gas generating agent used may range from about 0% to about 20% by weight of the compressed layer comprising swelling agents.

Examples of osmogents that may be used as swelling agent in the upper compressed layer, include, but are not limited to, sodium or potassium chloride, sodium or potassium hydrogen phosphate, sodium or potassium dihydrogen phosphate, salts of organic acids such as sodium or potassium acetate, magnesium succinate, sodium benzoate, sodium citrate or sodium ascorbate; carbohydrates such as mannitol, sorbitol, arabinose, ribose, xylose, glucose, fructose, mannose, galactose, sucrose, maltose, lactose, raffinose; water-soluble amino acids such as glycine, leucine, alanine, or methionine; urea and the like; a polymer consisting of acrylic acid lightly cross-linked with polyallylsucrose mixtures thereof. The amount of osmogents may vary from about 0% to about 20% by weight of the compressed layer comprising swelling agents.

In a preferred embodiment of the invention, the upper compressed layer or the bottom compressed layer comprises a swelling agent that is selected from cross linked polyvinyl pyrrolidone and cross linked carboxy methyl cellulose and a wicking agent for example, silicified microcrystalline cellulose.

The compressed layer comprising swelling agent additionally may comprise other excipients such as surfactants, lubricants, and other excipients commonly used in the pharmaceutical art. The upper compressed layer may optionally, include same or a different therapeutically active ingredient to cause a rapid release followed by a controlled release from the lower compressed layer comprising release rate controlling excipients. It may be noted that the upper compressed layer comprising swelling agent or the bottom compressed layer comprising swelling agents may have same of different composition.

The oral controlled release tablet of the present invention comprises a core and a coating surrounding the said core, the coating comprising a polymer insoluble in an aqueous medium of 0% v/v to 40% v/v alcohol content. The coating according to the present invention may be impermeable or semipermeable in nature. A coating is said to be impermeable when it does not allow permeation of both, the active ingredient and water whereas a coating is said to be semipermeable when it allows the permeation of water but does not allow permeation of the active ingredients. According to one embodiment, the coating comprises polymers that are impermeable. Upon disintegration of the compressed layer comprising swelling agents, the compressed layer comprising the active ingredient is exposed to the environment from only one surface thereby providing a defined surface area through which the release of the active ingredient takes place in a controlled manner, for example, zero order. In embodiments where the coating is semipermeable in nature, the permeation of only water can occur through the coating covering the surfaces of the tablet. Upon contact with aqueous environment, the compressed layer comprising the swelling agent swells and exerts pressure on the coating in its immediate vicinity and causes removal of the coating from that surface. The compressed layer of swelling agents disintegrates leaving behind the active ingredient compressed layer covered with coating on its other surfaces. As the coating is semi permeable, the release of the active ingredient is confined to this exposed surface only.

Examples of the polymer insoluble in an aqueous medium having 0% v/v to 40% v/v ethanol that are used in the coating according to present invention, includes, but are not limited to, ethyl cellulose, cellulose acetate, polyvinyl acetate, nitrocellulose, butadiene styrene copolymers, and water insoluble methacrylate copolymers. Preferably, the water insoluble polymer is selected from the group consisting of ethyl cellulose having ethoxy content of more than 46.5%, preferably in range of 48.0 to 49.5%, poly(ethyl acrylate, methyl methacrylate, triethylammonioethyl methacrylate chloride), in a ratio 1:2:0.1, (commercially available under the trade names Eudragit RS100, Eudragit RS PO, Eudragit RS 30D and Eudragit RS 12.5) and poly (ethyl acrylate, methyl methacrylate, trimethylammonioethymethacrylate chloride) in a ratio 1:2:0.2 (commercially available under the trade names Eudragit RL100, Eudragit RL PO, Eudragit RL 30D and Eudragit RL 12.5).

In one embodiment of the present invention, the water insoluble polymer included in the coating may be in the form of an aqueous dispersion. For example, aqueous dispersions of any of the aforementioned insoluble polymers may be used. Most preferably, an aqueous dispersion of ethyl cellulose is used.

Suitable aqueous dispersions of ethyl cellulose include those commercially available under the trade names Aquacoat ECD-30® from FMC Corporation (Philadelphia, USA) and Surelease® from Colorcon (West Point, Pa.). Aquacoat is an aqueous polymeric dispersion of ethylcellulose and contains sodium lauryl sulfate and cetyl alcohol, while Surelease® is an aqueous polymeric dispersion of ethyl cellulose and contains dibutyl sebacate, oleic acid, ammoniated water and fumed silica. The coat may be applied to a weight gain of about 5% to about 20% by weight, preferably from about 8% to about 15% by weight of the core.

According to the present invention, the coating may comprises one or more plasticizers. The plasticizers may be those that are conventionally used in the pharmaceutical art. These may be hydrophilic or hydrophobic in nature. Examples of hydrophilic plasticizer that may be used in the coating, include, but are not limited to, triethyl citrate, triethyl acetyl citrate, triacetin, tributyl citrate, polyethylene glycol 6000, polysorbate 80, glycerol and the like and mixtures thereof. Examples of hydrophobic plasticizer that may be used in the coating include, but are not limited to, dibutyl sebacate, diethyl sebacate, diethyl phthalate, vegetable and mineral oil, glyceryl tributyrate and the like and mixtures thereof. More preferably, the plasticizer is a mixture of hydrophobic and hydrophilic plasticizers. In a particular embodiment wherein the coating comprises ethyl cellulose as the polymer insoluble in 0% v/v to 40% v/v alcohol, the hydrophobic plasticizer is dibutyl sebacate and the hydrophilic plasticizer is triethyl citrate, preferably in the ratio of about 5:1. More particularly, the percent of dibutyl sebacate by weight of ethyl cellulose is about 5% and the percent of triethyl citrate is about 25% by weight of ethyl cellulose.

The coating of the controlled release tablet of the present invention, upon contact with the aqueous environment is removed from a surface of the said tablet by several means and the features. For example, the coating may have weakness created by either mechanical or electrical means, or by radiation, or by designing a brittle coating, or a thin coating, or a brittle and thin coating or a porous coating on the surfaces of the tablet which are in immediate vicinity of the compressed layer comprising swelling agents. The defect may also be instantly created on the said surface by leaching of components of the coating upon contact with the aqueous environment. The defect may also be in the form of an apparent fault such as an indent or a tear or a cut or an etching, which beginning from the outer surface of the coating may penetrate only partially through the coating or may penetrate completely to the inner surface of the coating so as to form a passageway. However, it is to be noted, that the defect in the form of tear or cut or etching or passageway does not expose the surface of the compressed layer comprising active ingredient and release rate controlling agent, to an appreciable extent. Particularly, an area of the coating is not removed in the process of creating a defect. The term “coating surrounding said core” includes such coatings that completely surround the core and may have tears or cuts or etchings or passageways without exposure of a significant area of the compressed layer of therapeutically active ingredient to the environment. For example, the term does not include a coating such as for example, the coating described in U.S. Pat. No. 5,560,169. Upon ingress of the aqueous environment, the upper compressed layer comprising swelling agent swells to cause removal of the coating from the upper surface of the tablet which lies in the immediate vicinity of the upper compressed layer. The upper compressed layer disintegrates allowing the release of the therapeutically active ingredient from the defined surface area of the compressed layer comprising therapeutically active ingredient and at least one release rate controlling excipient and wherein the total amount of excipients, that are soluble in alcohol, does not exceed 35% by weight of the said layer. This defined surface area of the said compressed layer remains substantially constant throughout the designed release period allowing release of the therapeutically active ingredient in a controlled manner.

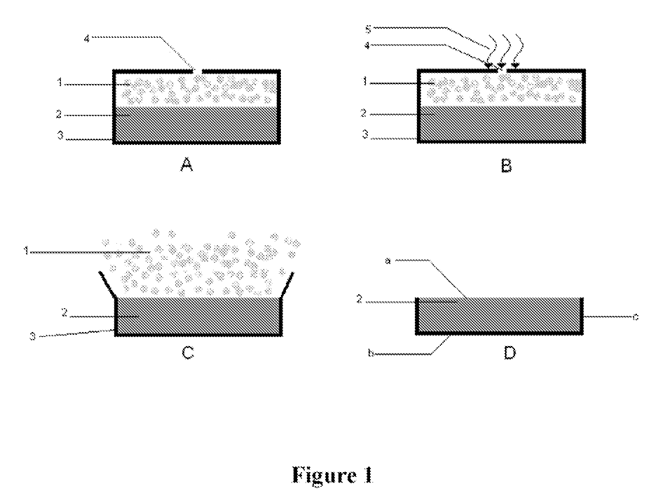

Specific embodiments where the coating has a preformed passageway, for example, laser drilled passageway, are illustrated in FIG. 1. In these embodiments, there is no substantial delay in the release of the active ingredient. The term “without a substantial delay” as used herein means that the active ingredient release is initiated from the oral controlled release tablet of the present invention within 0 to 60 minutes from the time the tablet contacts an aqueous environment, preferably within 0 to 20 minutes, and most preferably within 0 to 5 minutes. Such embodiments are described in our co-pending PCT application, WO2005039481 which is incorporated herein as reference.

Specific embodiment where the defect is created in situ by leaching of an enteric polymer in intestinal fluids is illustrated in FIG. 2 and FIG. 3. In these embodiments there is no release of the therapeutically active ingredient in the gastric fluids but upon contact with intestinal fluids, the enteric polymer becomes soluble and leaches out and release of the active ingredient occurs without substantially delay after contacting intestinal fluids. Such embodiments are described in our co-pending PCT application WO2006123364 which is incorporated herein.

Specific embodiment where the defect is created in situ by leaching of the leachable components of the coating in the gastrointestinal fluids is illustrated in FIG. 4 and FIG. 5. In these embodiments, there is no substantial delay in the release of the active ingredient. Such embodiments are described in our co-pending Indian patent of addition application, 2374/MUM/2007 which is incorporated herein as reference. In one embodiment of the present invention, the coating comprises a leachable component. The leachable component may be selected by a person of skill in the art from known water soluble substances and suitable amounts ordinarily determined by routine optimization. Accordingly in one embodiment of the present invention, the coating comprises one or more polymers that are insoluble in an aqueous medium having 0% v/v to 40% v/v alcohol, leachable components and other conventional coating additives such as plasticizers, colour and mixtures thereof and the like. Examples of leachable components that may be used include water soluble inorganic compounds and water soluble organic compounds. More specifically inorganic leachable compounds include, for example, sodium chloride, sodium bromide, sodium carbonate, potassium chloride, potassium sulfate, potassium phosphate, potassium nitrate, calcium phosphate, calcium nitrate, calcium chloride, and the like. More specifically, leachable organic compounds include, water soluble polymers such as water soluble cellulose polymers, polyols, for example polyhydric alcohol, polyalkylene glycol, polyglycol and the like. Organic compounds that may be used as leachable components also includes glucose, sucrose, sorbitol, mannitol, lactitol, lactose, sodium benzoate, sodium acetate, sodium citrate, low viscosity hydroxypropyl methyl cellulose, propylene glycol and the like. In one embodiment of the present invention, the coating on the core comprises ethyl cellulose and a mixture of mannitol and polyvinyl pyrrolidone.

According to one embodiment of the present invention, the coating surrounding the core of the controlled release tablet of the present invention is preferably impermeable to the therapeutically active ingredient, and has a pre formed passageway therein. In one preferred embodiment of the coating with a passageway, the coating is made up of water-insoluble polymers that may be selected from ethyl cellulose, hydrophobic methacrylic acid derivatives and the like, and mixtures thereof. A mechanically or laser-drilled passageway are made on the surface of the tablet which is in immediate vicinity of the compressed layer comprising swelling agent. Such embodiments are diagrammatically illustrated in FIG. 1 wherein the core is bilayer.

The following example does not limit the scope of the invention and are used as illustrations.

### EXAMPLE 1

Oral controlled release tablets were prepared using the ingredients listed in the Table 1 below.

TABLE 1

| | | % by weight |
|---|---|---|
| Ingredients | | of lower |
| Lower compressed layer | mgs per tablet | compressed layer |
| Metoprolol Succinate | 47.50 | 32.53 |
| Hydroxypropyl methyl cellulose | 20.0 | 13.69 |
| K100 M | | |
| Lactose directly compressible | 40.50 | 27.73 |
| Polyvinyl pyrrolidone | 10.0 | 6.85 |
| Eudragit L-100 55 | 10.0 | 6.85 |
| Hydroxypropyl methyl cellulose | 15.0 | 10.27 |
| K4 M | | |
| Aerosil | 0.50 | 0.34 |
| Talc | 1.25 | 0.85 |
| magnesium Stearate | 1.25 | 0.85 |
| | | % by weight |
| | | of the upper |
| Upper compressed layer | mgs per tablet | compressed layer |
| Silicified microcrystalline Cellulose | 63.042 | 79.80 |
| Colloidal Silicon Dioxide | 1.975 | 2.50 |
| Crospovidone | 11.85 | 15.00 |
| Sodium Lauryl Sulphate | 0.79 | 1.00 |
| FD&C Blue No. 1 Alu Lake | 0.316 | 0.40 |
| Magnesium Stearate | 0.9875 | 1.25 |
| Talc | 0.1975 | 0.25 |

| Coating composition | mg per tablet |
|---|---|
| *Ethyl cellulose | 10.12 |
| *Celyl alcohol | 0.37 |
| *Sodium lauryl sulphate | 0.74 |
| Triethyl citrate | 0.56 |
| Dibutyl sebacate | 2.81 |

The amount of alcohol soluble excipients in the upper compressed layer i.e, polyvinyl pyrrolidone and Eudragit L-100 55 is about 13.5% by weight of the upper compressed layer.

Metoprolol succinate, hydroxypropylmethyl cellulose, lactitol monohydrate and povidone K-30 were passed through ASTM (American Society for Testing and Materials) sieve #40 and mixed suitably. The mixture thus obtained was granulated with purified water to a suitable end-point, and the granules obtained were dried to a moisture content of about 1-2%. The dried granules were milled suitably and lubricated with a mixture of sodium starch glycolate, colloidal silicon dioxide, talc and magnesium stearate, to obtain the blend for the lower compressed layer.

Silicified microcrystalline cellulose, crospovidone, sodium lauryl sulfate and a suitable colour were passed through ASTM sieve #40 and mixed suitably. The blend so obtained was lubricated with a mixture of colloidal silicon dioxide and magnesium stearate (previously passed through ASTM sieve #60). The above two preparations were compressed to obtain bilayer tablets, which were coated with an aqueous dispersion of ethyl cellulose to a suitable weight gain. An orifice was then drilled on the surface of the tablet having in its immediate vicinity the upper compressed layer.

The tablet of example 1 upon contact with aqueous environment behaves in a manner as illustrated in FIG. 1. Upon contact of the coated tablet with the aqueous fluids, there is a rapid ingress of fluids through the passageway. The silicified microcrystalline cellulose of the upper compressed layer facilitates a rapid ingress of water. The superdisintegrant, namely crospovidone imbibes water and swells and exerts pressure from inside of the coating surrounding the upper surface of the tablet as diagrammatically represented in FIG. 1(B). This causes the coating to be removed from the upper surface exposing the upper compressed layer to the aqueous environment. This layer then completely disintegrated as represented in FIG. 1(C). The lower compressed layer comprising metoprolol succinate and other release rate controlling excipients are exposed to a defined surface area from where the release of the drug takes place as illustrated diagrammatically in FIG. 1(D).

The upper surface of the lower compressed layer along the length of the tablet is exposed to the aqueous environment. The coating is retained on the bottom and side surfaces of the lower compressed layer. This coating being impermeable to metoprolol succinate, the release of metoprolol succinate occurs essentially through a substantially unchanged surface area of the upper surface of the lower compressed layer over the period of release. The lower compressed layer on exposure to water forms a gel and slowly releases metoprolol succinate. Without being bound to any theory, we believe the release in this embodiment may be occurring through a combined mechanism of partial erosion and diffusion. In this embodiment illustrated by this example, in which the lower compressed layer used a high molecular weight grades of hydroxypropyl methyl cellulose in amounts greater than about 15% by weight of the lower compressed layer, at the end of the release period, lower compressed layer releases the active ingredient completely leaving behind a three sided coating, herein referred to as ‘cup’ with the semi rigid hydrogel matrix inside the ‘cup’

### EXAMPLE 2

The oral controlled release tablets comprising paroxetine hydrochloride were obtained as per the present invention, as detailed in Table 2 below.

TABLE 2

| | Quantity | |
|---|---|---|
| | | % w/w of |
| Ingredients | mg/tablet | the layer |

Lower compressed layer

| | | |
|---|---|---|
| Paroxetine hydrochloride hemihydrate | 42.66 | 24.38 |
| (equivalent to Paroxetine base 37.5 mg) | | |
| Hydroxypropyl methylcellulose (Methocel | 40.00 | 22.86 |
| K100LV) | | |
| Polyvinylpyrrolidone (Povidone K-30) | 10.00 | 5.71 |
| Lactose monohydrate | 52.31 | 29.91 |
| Silicified microcrystalline cellulose (Prosolv | 27.00 | 15.43 |
| SMCC) | | |
| Colloidal silicon dioxide | 1.00 | 0.57 |
| Magnesium stearate | 2.00 | 1.14 |

Upper compressed layer

| | | |
|---|---|---|
| Silicified microcrystalline cellulose (Prosolv | 84.8 | 84.8 |
| SMCC) | | |
| Crospovidone | 10.0 | 10.0 |
| Colloidal silicon dioxide | 2.5 | 2.5 |
| Sodium lauryl sulfate | 1.0 | 1.0 |
| Color (FD&C blue lake no 1) | 0.4 | 0.4 |
| Magnesium stearate | 1.05 | 1.05 |
| Talc | 0.25 | 0.25 |

Coating

| | | |
|---|---|---|
| Aquacoat ECD 30 solids (aqueous | 21.34 | Coated to a |
| ethyl cellulose dispersion) | | weight gain of |
| Acryl eze white 9318509 | 11.75 | about 12% by |
| Dibutyl sebacate | 1.60 | weight of the |
| Triethyl citrate | 0.64 | bilayered core |

Paroxetine hydrochloride hemihydrate, hydroxypropyl methyl cellulose, lactose monohydrate and povidone K-30 (alcohol soluble excipient in amount of about 5.71% by weight of the lower compressed layer) were passed through ASTM (American Society for Testing and Materials) sieve # 40 and mixed suitably. The mixture thus obtained was granulated with purified water to a suitable end-point, and the granules obtained were dried to a moisture content of about 1-2%. The dried granules were milled suitably and lubricated with a mixture of Prosolv SMCC 90, colloidal silicon dioxide and magnesium stearate, to obtain the blend for the first layer. Silicified microcrystalline cellulose, crospovidone, sodium lauryl sulfate and a suitable color were passed through ASTM sieve #40 and mixed suitably. The blend so obtained was lubricated with a mixture of colloidal silicon dioxide, talc and magnesium stearate (previously passed through ASTM sieve #60).

The above two preparations were compressed to obtain bilayer tablets, which were coated with an aqueous dispersion containing ethyl cellulose, Acryl-Eze, dibutyl sebacate and triethyl citrate to a weight gain of about 12% by weight of the core.

The tablet of example 2 upon contact with aqueous environment behaves in a manner as illustrated in FIG. 2. Upon contact with the gastric acidic fluid, the tablet does not release any drug as the enteric polymer, namely, Acryl Eze is not soluble in acidic media. Upon contact with the alkaline intestinal fluids, the enteric polymer dissolves which creates microporous channels in the coating as illustrated in FIG. 2(C). A rapid ingress of fluids occurs through these micro pores. The upper compressed layer comprising silicified microcrystalline cellulose facilitates a rapid ingress of water. The superdisintegrant, namely crospovidone imbibes water, swells and exerts pressure from inside of the coating located at the upper surface of the tablet causing removal of the coating from that surface. After this, the upper compressed layer is completely disintegrated as represented in FIG. 2(D). The lower compressed layer comprising paroxetine hydrochloride and other release rate controlling excipients are exposed to a defined surface area from where the release of the drug takes place, as illustrated diagrammatically in FIG. 2(E).

The lower compressed layer starts gelling upon contact with the aqueous environment. The coating is retained on the bottom and side surfaces of the lower compressed layer. The coating is substantially impermeable to paroxetine hydrochloride. The release occurs from the exposed upper surface of the gelled lower compressed layer. The surface area of the release remains substantially constant over the period of release. In this embodiment, it is observed that as the release occurs, the lower compressed layer erodes and its thickness reduces until the cup formed by the coating is emptied completely at the end of the release period.

### EXAMPLE 3

The bilayer core comprising a upper compressed layer and a lower compressed layer comprising active ingredient having the ingredients as given in table 3, are prepared as follows.

TABLE 3
Bilayer core of the tablet

| | | % by weight |
|---|---|---|
| Ingredients of upper compressed | | of the upper |
| layer | mg per tablet | compressed layer |
| Silicified microcrystalline cellulose | 105.728 | 80.096 |
| Colloidal Silicon Dioxide | 3.30 | 2.50 |
| Crospovidone | 19.8 | 15.0 |
| Sodium lauryl sulphate | 1.32 | 1.0 |
| FD & C Blue No. 1 Aluminum lake | 0.568 | 0.43 |
| Magnesium stearate | 1.39 | 1.053 |
| talc | 0.33 | 0.25 |
| | | % by weight of the |
| | | lower compressed |
| Ingredients of lower compressed | | layer of active |
| layer | mg per tablet | ingredient |
| Venlafaxine hydrochloride | 169.710 | 36.57 |
| Hydroxypropyl methyl cellulose | 33.00 | 7.11 |
| K4M | | |
| Polyvinyl pyrrolidone | 40.00 | 8.62 |
| Lactose monohydrate | 175.290 | 37.78 |
| Eudragit L100 55 | 60.0 | 8.62 |
| Talc | 3.0 | 0.64 |
| Magnesium stearate | 3.0 | 0.64 |

The bilayer core is then coated with the coating composition, details of which are given in table 4.

TABLE 4
coating composition

| Bilayer core | As in table 3 |
|---|---|
| Coating composition | |
| *Ethyl cellulose | 26.73 |
| * Cetyl alcohol | 0.99 |
| * Sodium lauryl sulphate | 1.98 |
| Triethyl citrate | 1.486 |
| Dibutyl sebacate | 7.428 |
| Polyvinyl pyrrolidone | 1.783 |
| mannitol | 7.131 |

applied in the form of Aquacoat ECD 30 which is an aqueous dispersion containing ethyl cellulose (27% w/v), sodium lauryl sulphate 1% w/w and cetyl alcohol 2% w/v in water

The tablet of example 3, upon contact with aqueous environment behaves in a manner as illustrated in FIG. 4. Upon contact with the aqueous fluids, the water soluble leachable components, namely, mannitol and polyvinyl pyrrolidone dissolve which creates microporous channels in the coating. A rapid ingress of fluids occurs through these micro pores. The upper compressed layer comprising silicified microcrystalline cellulose facilitates a rapid ingress of water. The superdisintegrant, namely crospovidone imbibes water, swells and exerts pressure from inside of the coating located at the upper surface of the tablet causing removal of the coating from that surface. After this, the upper compressed layer is completely disintegrated as represented in FIG. 4(D). The lower compressed layer comprising venlafaxine hydrochloride and other release rate controlling excipients are exposed to a defined surface area from where the release of the drug takes place, as illustrated diagrammatically in FIG. 4(E).

The lower compressed layer starts gelling upon contact with the aqueous environment. The coating is retained on the bottom and side surfaces of the lower compressed layer. The costing is substantially impermeable to venlafaxine hydrochloride. The release occurs from the exposed upper surface of the gelled lower compressed layer. The surface area of the release remains substantially constant over the period of release. In this embodiment, it is observed that as the release occurs, the lower compressed layer erodes and its thickness reduces until the cup formed by the coating is emptied completely at the end of the release period.

### EXAMPLE 4

The upper compressed layer and the lower compressed layers contents and its amounts are given in table 5. Metoprolol succinate, hydroxypropyl methyl cellulose, lactose, povidone and Eudragit E were mixed and granulated. The granules were dried. The dried granules of the drug composition were mixed with Eudragit L-100-55 and the blend was lubricated with talc, magnesium stearate and colloidal silicon dioxide. The ingredients of the swelling composition was mixed and converted into slugs. The slugs of the swelling composition (62 mg) and the drug composition blend (138 mg) were compressed together to get bilayer core.

The bilayer core is coated with a coating composition as given in table 6. The coated tablets are further coated with Opadry coating to a weight gain of about 3%.

TABLE 5
Bilayer core composition

| | | % by |
|---|---|---|
| Ingredients of | | weight of upper |
| Upper composition layer | mgs per tablet | compressed layer |
| Silicified microcrystalline Cellulose | 49.6 | 79.32 |
| Colloidal Silicon Dioxide | 1.55 | 2.48 |
| Crospovidone | 9.3 | 14.87 |
| Sodium Lauryl Sulphate | 0.62 | 0.991 |
| FD&C Blue No. 1 Alu Lake | 0.651 | 1.04 |
| Magnesium Stearate | 0.651 | 1.04 |
| Talc | 0.155 | 0.248 |
| Total | 62.53 | 100 |
| | | % by |
| | | weight of lower |
| Lower compressed layer | mg per tablet | compressed layer |
| metoprolol Succinate | 47.50 | 34.42 |
| equivalent to Metoprolol tartarate | | |
| 50 mg | | |
| Hydroxypropyl methyl cellulose | 10.0 | 7.45 |
| (viscosity 80,000-120000) | | |
| Lactose directly compressible | 20.50 | 14.86 |
| Polyvinyl pyrrolidone | 15.0 | 0.793 |
| Eudragit E | 20.0 | 14.49 |
| Eudragit L-100-55 | 22.0 | 15.94 |
| Aerosil | 0.50 | 0.362 |
| Talc | 1.25 | 0.906 |
| Magnesium Stearate | 1.25 | 0.906 |
| Total | 138.0 | 100 |

TABLE 6
Coating composition

| Ingredients | mg per tablet |
|---|---|
| Bilayer core | As given in table 5 |
| *Ethyl cellulose | 9.58 |
| *Cetyl alcohol | 0.71 |
| *Sodium lauryl sulphate | 0.355 |
| Mannitol | 0.786 |
| Triethyl citrate | 0.54 |
| Dibutyl sebacate | 2.66 |
| Hydroxypropyl methyl cellulose (6 cps) | 0.318 |
| Polyethylene glycol 400 | 0.0159 |
| Polyethylene glycol 8000 | 0.0159 |
| Polysorbate 20 | 0.05 |
| % weight gain | 8 |

*applied in the form of Aquacoat ECD 30 which is an aqueous dispersion containing ethyl cellulose (27% w/v), sodium lauryl sulphate 1% w/w and cetyl alcohol 2% w/v in water

The amount of alcohol soluble excipients, namely, polyvinyl pyrrolidone, Eudragit E and Eudragit L-500 55 is about 31.22% by weight of the compressed layer of the active ingredient with release rate controlling excipients.

The tablet of example 4, upon contact with aqueous environment behaves in a manner as illustrated in FIG. 4. Upon contact with the aqueous fluids, the water soluble leachable components, namely, mannitol and polyvinyl pyrrolidone dissolve which creates micro-porous channels in the coating. A rapid ingress of fluids occurs through these micro pores. The upper compressed layer comprising silicified microcrystalline cellulose facilitates a rapid ingress of water. The super-disintegrant, namely crospovidone imbibes water, swells and exerts pressure from inside of the coating located at the upper surface of the tablet causing removal of the coating from that surface. After this, the upper compressed layer is completely disintegrated as represented in FIG. 4(D). The lower compressed layer comprising metoprolol succinate and other release rate controlling excipients are exposed to a defined surface area from where the release of the drug takes place, as illustrated diagrammatically in FIG. 4(E).

The release of the drug takes place in a manner similar to example 1, but the lower compressed layer is more rigid compared to the lower compressed layer of example 1. This is because of the presence of Eudragit E which is insoluble in phosphate buffer. At the end of the release period, a rigid, rubbery matrix is seen inside the cup formed by the coating is emptied completely at the end of the release period.

### EXAMPLE 5

Tablets prepared according to the example 3 were tested in vitro using type I basket apparatus rotating at a speed of 100 rpm at 37° C. in 900 ml of dissolution media having phosphate buffer pH 6.8 with and without 40% ethanol. The release of the drug was monitored for 24 hours. The dissolution details are tabulated in Table 7 and the graph of % dissolution Vs time in hours in presented in FIG. 6. The dissolution of the tablets of Example 3 was compared with Venlafaxine capsules commercially available under the brand name of Effexor® XR by Wyeth.

TABLE 7
In vitro dissolution in phosphate buffer
with and without 40% v/v ethanol

| | % Venlafaxine released in 900 ml of phosphate buffer PH 6.8 | | | |
|---|---|---|---|---|
| | with 40% v/v ethanol | | Without 40% v/v ethanol | |
| Time | Example 3 of | | Example 3 of | |
| in | the present | Effexor ER ® | the present | Effexor ER ® |
| hours | invention | capsules | invention | capsules |
| 0 | 0 | 0 | 0 | 0 |
| 1 | 10 | 52 | 7 | 5 |
| 2 | 17 | 80 | 13 | 16 |
| 4 | 33 | 90 | 24 | 40 |
| 6 | 53 | 100 | 34 | 56 |
| 8 | 66 | 101 | 46 | 66 |
| 10 | 82 | 101 | 56 | 72 |
| 12 | 89 | 100 | 66 | 77 |
| 14 | 89 | 100 | — | — |
| 18 | 92 | 99 | — | — |
| 20 | 90 | 97 | 89 | 87 |
| 24 | 90 | 96 | — | 89 |

The tablets of example 3 were also subjected to intro dissolution testing in 0.1 N HCL with 40% ethanol. The release of the drug was monitored for 2 hours at the specified time points mentioned in table 8.

TABLE 8
In vitro dissolution of drug of Example 3 in
0.1 N HCL in 40% v/v ethanol

| Time in | % dissolution of Venlafaxine in 0.1 N |
|---|---|
| minutes | HCL in 40% v/v ethanol |
| 15 | 0 |
| 30 | 3 |
| 45 | 5 |
| 60 | 7 |
| 75 | 8 |
| 90 | 10 |
| 105 | 11 |
| 120 | 13 |

It is observed that the oral controlled release tablet of the present invention reduces the risk of alcohol-induced dose-dumping as illustrated by the % dissolution of the said tablets in 40% v/v alcohol.

### EXAMPLE 6

Tablets prepared according to the example 4 were tested in vitro using type I basket apparatus rotating at a speed of 100 rpm at 37° C. in 900 ml of dissolution media having phosphate buffer pH 6.8 with and without 40% ethanol. The release of the drug was monitored for 24 hours. The dissolution details are tabulated in Table 9 and the graph of % dissolution Vs time in hours in presented in FIG. 7.

TABLE 9
In vitro dissolution in phosphate buffer
with and without 40% v/v ethanol

| | |
|---|---|
| | % metoprolol released in 900 ml |
| Time in | of phosphate buffer PH 6.8 |

| hours | with 40% v/v ethanol | without 40% v/v ethanol |
|---|---|---|
| 0 | 0 | 0 |
| 1 | 7 | 5 |
| 2 | 15 | 9 |
| 4 | 38 | 16 |
| 6 | 63 | 25 |
| 8 | 83 | 33 |
| 10 | 94 | — |
| 12 | 99 | 47 |
| 14 | 99 | — |
| 18 | 100 | — |
| 20 | 101 | 69 |
| 24 | 99 | 77 |

The tablets of example 4 were also subjected to dissolution testing in 0.1 N HCL with 40% ethanol. The release of the drug was monitored for 2 hours at the specified time points mentioned in table 10.

TABLE 10
In vitro dissolution of drug of Example 4 and Toprol ® XL in 0.1 N HCL with varying
amounts of ethanol in type II (paddle apparatus)

| | % dissolution of Metoprolol in 0.1 N HCL having 5%, 20% and 40% alcohol | | | | | | | |
|---|---|---|---|---|---|---|---|---|
| | 0% alcohol | | 5% alcohol | | 20% alcohol | | 40% alcohol | |
| Time in | | Toprol ® | | Toprol ® | | Toprol ® | | Toprol ® |
| mins | Example 4 | XL | Example 4 | XL | Example 4 | XL | Example 4 | XL |
| 15 | 0 | 3 | 1 | 4 | 0 | 3 | 1 | 4 |
| 30 | 3 | 7 | 3 | 7 | 1 | 10 | 2 | 23 |
| 45 | 6 | 9 | 5 | 11 | 3 | 16 | 4 | 50 |
| 60 | 8 | 12 | 7 | 14 | 4 | 26 | 5 | 68 |
| 75 | 10 | 15 | 9 | 17 | 6 | 37 | 6 | 77 |
| 90 | 12 | 17 | 11 | 20 | 7 | 48 | 8 | 83 |
| 105 | 14 | 19 | 12 | 24 | 8 | 58 | 9 | 86 |
| 120 | 17 | 22 | 14 | 28 | 9 | 67 | 10 | 89 |

Toprol ® XL is a product available in the market in the form of pellets compressed into tablets

It is observed that the oral controlled release tablet of the present invention reduces the risk of alcohol-induced dose-dumping as illustrated by the % dissolution of the said tablets in 5% v/v to 40% v/v alcohol.

### EXAMPLE 7

Oral controlled release tablets were prepared using the ingredients listed in the Table 11 below.

TABLE 11

| | | % by weight |
|---|---|---|
| Ingredients | | of lower |
| Lower compressed layer | mgs per tablet | compressed layer |
| Metoprolol Succinate | 95 | 49.6 |
| Hydroxypropyl methyl cellulose | 13.5 | 7.04 |
| K100 M | | |
| Lactose directly compressible | 10 | 5.22 |
| Polyvinyl pyrrolidone | 17.5 | 9.13 |
| Eudragit EPO | 13 | 6.78 |
| Eudragit L100-55 | 32.5 | 16.97 |
| Aerosil | 1.5 | 0.78 |
| Talc | 4.25 | 2.21 |
| magnesium Stearate | 4.25 | 2.21 |
| | | % by weight |
| | | of the upper |
| Upper compressed layer | mgs per tablet | compressed layer |
| Silicified microcrystalline Cellulose | 41.65 | 80.49 |
| Colloidal Silicon Dioxide | 1.3 | 2.51 |
| Crospovidone | 7.8 | 15.07 |
| Sodium Lauryl Sulphate | 0.52 | 1.0 |
| FD&C Blue No. 1 Alu Lake | 0.05 | 0.09 |
| Magnesium Stearate | 0.55 | 1.06 |
| Talc | 0.13 | 0.25 |

| Coating composition | mg per tablet |
|---|---|
| *Ethyl cellulose | 12.13 |
| *Cetyl alcohol | 0.9 |
| *Sodium lauryl sulphate | 0.45 |
| Mannitol | 0.67 |
| Povidone K-30 | 0.102 |
| Triethyl citrate | 0.24 |
| Dibutyl sebacate | 1.215 |
| Purified talc | 0.79 |

*applied in the form of Aquacoat ECD 30 which is an aqueous dispersion containing ethyl cellulose (27% w/v), sodium lauryl sulphate 1% w/w and cetyl alcohol 2% w/v in water

The amount of alcohol soluble excipients in the upper compressed layer i.e, polyvinyl pyrrolidone and Eudragit L-100 55 is about 13.5% by weight of the upper compressed layer. Metoprolol succinate, hydroxypropylmethyl cellulose, lactitol monohydrate and povidone K-30 were passed through ASTM (American Society for Testing and Materials) sieve #40 and mixed suitably. The mixture thus obtained was granulated with purified water to a suitable end-point, and the granules obtained were dried to a moisture content of about 1-2%. The dried granules were milled suitably and lubricated with a mixture of sodium starch glycolate, colloidal silicon dioxide, talc and magnesium stearate, to obtain the blend for the lower compressed layer.

Silicified microcrystalline cellulose, crospovidone, sodium lauryl sulfate and a suitable colour were passed through ASTM sieve #40 and mixed suitably. The blend so obtained was lubricated with a mixture of colloidal silicon dioxide and magnesium stearate (previously passed through ASTM sieve #60). The above two preparations were compressed to obtain bilayer tablets, which were coated with an aqueous dispersion of ethyl cellulose to a suitable weight gain.

TABLE 12
In vitro dissolution in phosphate buffer with
and without 40% v/v ethanol

| | |
|---|---|
| | % metoprolol released in 900 ml |
| Time in | of phosphate buffer PH 6.8 |

| hours | with 40% v/v ethanol | without 40% v/v ethanol |
|---|---|---|
| 0 | 0 | 0 |
| 1 | 5 | 3 |
| 2 | 11 | 8 |
| 4 | 19 | 14 |
| 6 | 26 | 23 |
| 8 | 32 | 30 |
| 10 | 38 | 37 |
| 12 | 43 | 44 |
| 14 | 51 | 57 |
| 20 | 59 | 69 |
| 24 | 66 | 77 |

### EXAMPLE 8

Oral controlled release tablets were prepared using the ingredients listed in the Table 13 below.

TABLE 13

| | | % by weight |
|---|---|---|
| Ingredients | | of lower |
| Lower compressed layer | mgs per tablet | compressed layer |
| Metoprolol Succinate | 190 | 49.6 |
| Hydroxypropyl methyl cellulose | 27 | 7.04 |
| K100 M | | |
| Lactose directly compressible | 20 | 5.22 |
| Polyvinyl pyrrolidone | 35 | 9.14 |
| Eudragit EPO | 26 | 6.78 |
| Eudragit L100-55 | 65 | 16.97 |
| Aerosil | 3 | 0.78 |
| Talc | 8.5 | 2.21 |
| magnesium Stearate | 8.5 | 2.21 |
| | | % by weight |
| | | of the upper |
| Upper compressed layer | mgs per tablet | compressed layer |
| Silicified microcrystalline Cellulose | 83.3 | 80.06 |
| Colloidal Silicon Dioxide | 2.6 | 2.5 |
| Crospovidone | 15.6 | 14.99 |
| Sodium Lauryl Sulphate | 1.04 | 0.99 |
| FD&C Blue No. 1 Alu Lake | 0.1 | 0.096 |
| Magnesium Stearate | 0.84 | 0.80 |
| Talc | | |

| Coating composition | mg per tablet |
|---|---|
| *Ethyl cellulose | 21.01 |
| *Cetyl alcohol | 1.556 |
| *Sodium lauryl sulphate | 0.778 |
| Mannitol | 4.1 |
| Povidone K-30 | 0.512 |
| Triethyl citrate | 1.167 |
| Dibutyl sebacate | 5.836 |
| Polysorbate 20 | 0.102 |
| Purified talc | 3.896 |

*applied in the form of Aquacoat ECD 30 which is an aqueous dispersion containing ethyl cellulose (27% w/v), sodium lauryl sulphate 1% w/w and cetyl alcohol 2% w/v in water

The amount of alcohol soluble excipients in the upper compressed layer i.e, polyvinyl pyrrolidone and Eudragit L-100 55, Eudragit EPO is about 32.89% by weight of the upper compressed layer. Metoprolol succinate, hydroxypropylmethyl cellulose, lactose were passed through ASTM (American Society for Testing and Materials) sieve #40 and mixed suitably. The mixture thus obtained was granulated with purified water to a suitable end-point, and the granules obtained were dried to a moisture content of about 1-2%. The dried granules were milled suitably and lubricated with a mixture of sodium starch glycolate, colloidal silicon dioxide, talc and magnesium stearate, to obtain the blend for the lower compressed layer.

Silicified microcrystalline cellulose, crospovidone, sodium lauryl sulfate and a suitable colour were passed through ASTM sieve #40 and mixed suitably. The blend so obtained was lubricated with a mixture of colloidal silicon dioxide and magnesium stearate (previously passed through ASTM sieve #60). The above two preparations were compressed to obtain bilayer tablets, which were coated with an aqueous dispersion of ethyl cellulose to a suitable weight gain.

TABLE 14
In vitro dissolution in phosphate buffer with
and without 40% v/v ethanol

| | % metoprolol released in 900 ml | |
|---|---|---|
| Time in | of phosphate buffer PH 6.8 | |
| minutes | with 40% v/v ethanol | without 40% v/v ethanol |
| 0 | 0 | 0 |
| 15 | 0 | 1 |
| 30 | 0 | 3 |
| 45 | 2 | 4 |
| 60 | 3 | 6 |
| 75 | 4 | 7 |
| 90 | 5 | 8 |
| 102 | 6 | 9 |
| 120 | 6 | 10 |

1. A method of reducing the risk of alcohol-induced dose-dumping of a therapeutically active ingredient comprising administering to human subjects who have ingested alcohol an oral controlled release tablet; said tablet comprising:
a core comprising
an upper compressed layer comprising a swelling agent, and
a lower compressed layer comprising at least one therapeutically active ingredient, and pharmaceutically acceptable excipient wherein at least one excipient is a release rate controlling excipient and wherein the percent by weight of excipients that are soluble in alcohol does not exceed 35% by weight of the layer and;
a coating surrounding the said core, the coating comprising a polymer insoluble in an aqueous medium comprising from 0% v/v to 40% v/v of alcohol,
whereby upon contact with aqueous gastrointestinal fluids, the upper compressed layer swells to cause removal of the coating from the upper surface of the upper compressed layer and then said upper layer disintegrates allowing the release of the active ingredient from the defined surface area of the upper surface of said lower compressed layer with the coating covering its bottom and side surfaces.

2. A method of reducing the risk of alcohol-induced dose-dumping of a therapeutically active ingredient comprising administering to human subjects who have ingested alcohol an oral controlled release tablet; said tablet comprising:
a core comprising
an upper compressed layer comprising a swelling agent, and
a middle compressed layer comprising at least one therapeutically active ingredient, and pharmaceutically acceptable excipient wherein at least one excipient is a release rate controlling excipient and wherein the total amount of excipients that are soluble in alcohol does not exceed 35% by weight of the layer and;
a bottom compressed layer comprising a swelling agent,
a coating surrounding the said core, the coating comprising a polymer insoluble in an aqueous medium comprising from 0% v/v to 40% v/v of alcohol content,
whereby upon contact with aqueous gastrointestinal fluids, the upper compressed layer and the bottom compressed layer swell to cause removal of the coating from the upper surface of the upper compressed layer and the lower surface of the bottom compressed layer and then said upper layer and the said bottom layer disintegrate, allowing the release of the active ingredient from the defined surface area of the upper and lower surface of said middle compressed layer with the coating covering its side surfaces.

3. A method as claimed in 1 or 2 wherein the swelling agent is selected from the group comprising super-disintegrants, wicking agents, osmogents, gas generating agents and mixtures thereof.

4. A method as claimed in claim 1 wherein the wicking agent in present in amounts ranging from about 5% to about 80% by weight of the upper compressed layer.

5. A method as claimed in claim 2 wherein the wicking agent in present in amounts ranging from about 5% to about 80% by weight of the upper and bottom compressed layers.

6. A method as claimed in claim 1 wherein the super-disintegrant is present in amount ranging from about 5% to about 30% by weight of the upper compressed layer.

7. A method as claimed in claim 2 wherein the super-disintegrant is present in amount ranging from about 5% to about 30% by weight of the upper and lower compressed layers.

8. A method as claimed in claim 1 or claim 2 wherein the coating is impermeable or semi-permeable to the therapeutically active ingredient.

9. A method as claimed in claim 1 or claim 2 wherein the coating comprises one or more leachable component.

10. A method as claimed in claim 1 or claim 2 the coating has one or more pre formed passageways located in the immediate vicinity of the compressed layer comprising swelling agent.

11. A method as claimed in claim 1 or claim 2 wherein the total amount of excipients that are soluble in alcohol does not exceed 25% by weight of the lower compressed layer.

12. A method as claimed in claim 1 wherein the total amount of excipients that are soluble in alcohol does not exceed 25% by weight of the middle compressed layer.

13. A method as claimed in claim 1 or claim 2 wherein the oral controlled release tablet does not release more than 80% of the active ingredient in about 4 hours when tested in vitro in 40% v/v ethanol.

14. A method as claimed in claim 1 or claim 2 the oral controlled release tablet does not release more than 40% of the active ingredient in about 2 hours when tested in vitro in 40% v/v ethanol.

15. A method as claimed in claim 1 or claim 2 wherein polymer insoluble in an aqueous medium of 0% v/v to 40% v/v of alcohol content is ethyl cellulose.

16. A method as claimed in claim 1 or claim 2 wherein the ethyl cellulose has ethoxy content is more than 46.5%.

17. A method as claimed in claim 1 or claim 2 wherein the ethyl cellulose has ethoxy content ranging from about 48% to about 49.5%.
